SEQUENCE LISTING (8 sequences)

SEQ ID NO: 1 - DNA, 8658 bp, unidentified
  source 1..8658: mol_type = genomic DNA
ctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaacgcatacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccgcatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatgcaaacaattaccccgacaaaggcgtggtggaaatcaaggccctgcctgagatcgccatgagcatcaagaaggcctttggggagtacgccatcatgaagggaagcctgcaggcggccgctcgagcctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgtt

SEQ ID NO: 2 - DNA, 3063 bp, unidentified
  source 1..3063: mol_type = genomic DNA
agtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacgcaggaaacagctatgaccatgattacgccagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccatcttgtggaaaggacgaaacaccgaaaaaaaaaaaaaaaaaaaaaaaaaaaaaagctaaaaatgtatatgcccaaagcaagttgtggaaggtccagttttatgtcttcctgggacgaagacaagttgtggaaggtccagttttgaggggctattacaacttttttggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttacc

SEQ ID NO: 3 - DNA, 3058 bp, unidentified
  source 1..3058: mol_type = genomic DNA
agtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacgcaggaaacagctatgaccatgattacgccagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaaaaaaaaaaaaaaaaaaaaaaaaaaaaaaaaaaaaaaaaaaaaaaaaaagctaaaaatgtatatgcccaaagcaagttgtggaaggtccagttttatgtcttcctgggacgaagacaagttgtggaaggtccagttttgaggggctattacaacttttttggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttacc

SEQ ID NO: 4 - DNA, 3083 bp, unidentified
  source 1..3083: mol_type = genomic DNA
agtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacgcaggaaacagctatgaccatgattacgccagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaaaaaaaaaaaaaaaaaaaaaaaaaaaaaaaaaaaaaaaaaaaaaaaaaaaaaaaaaaaaaaaaaaaaaaaaaaagctaaaaatgtatatgcccaaagcaagttgtggaaggtccagttttatgtcttcctgggacgaagacaagttgtggaaggtccagttttgaggggctattacaacttttttggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttacc

SEQ ID NO: 5 - DNA, 30 bp, unidentified
  source 1..30: mol_type = genomic DNA
aaaaaaaaaaaaaaaaaaaaaaaaaaaaaa

SEQ ID NO: 6 - DNA, 50 bp, unidentified
  source 1..50: mol_type = genomic DNA
aaaaaaaaaaaaaaaaaaaaaaaaaaaaaaaaaaaaaaaaaaaaaaaaaa

SEQ ID NO: 7 - DNA, 75 bp, unidentified
  source 1..75: mol_type = genomic DNA
aaaaaaaaaaaaaaaaaaaaaaaaaaaaaaaaaaaaaaaaaaaaaaaaaaaaaaaaaaaaaaaaaaaaaaaaaaa

SEQ ID NO: 8 - DNA, 23 bp, unidentified
  source 1..23: mol_type = genomic DNA
gctaaaaatgtatatgcccaaag

(19) United States
(12) Patent Application Publication
(10) Pub. No.: US 2026/0248960 A1
(43) Pub. Date: Aug. 27, 2026

(54) SYSTEMS FOR ENHANCING MRNA EXPRESSION AND USES THEREOF

(71) Applicant: The Johns Hopkins University, Baltimore, MD (US)

(72) Inventors: Jeffery Coller, Frederick, MD (US); Bahareh Torkzaban, Jessup, MD (US)

(21) Appl. No.: 18/861,531

(22) PCT Filed: Apr. 28, 2023
(86) PCT No.: PCT/US2023/066354

§ 371 Date: Oct. 29, 2024

Related U.S. Application Data
(60) Provisional application No. 63/336,656, filed on Apr. 29, 2022.

(51) Int. Cl. A61K 48/00; A61K 38/17; A61P 25/28; C12N 9/22; C12N 15/11; C12N 15/86

(52) U.S. Cl. CPC A61K 48/005; A61K 38/1709; A61P 25/28; C12N 9/226; C12N 15/111; C12N 15/86; C12N 2310/20; C12N 2710/10043; C12N 2740/15043; C12N 2750/14143; C12N 2830/50

(57) ABSTRACT

Provided herein are systems comprising: (a) an RNA binding moiety; and (b) a gRNA hybrid comprising (i) a complementary region that hybridizes with an mRNA of an active allele of a gene associated with a disorder associated with a decrease in the expression of a protein from the mRNA and (ii) a poly(A) tail, wherein the gRNA hybrid forms a complex with the RNA binding moiety.

### CROSS-REFERENCE TO RELATED APPLICATIONS

This application claims priority to U.S. Provisional Patent Application No. 63/336,656, filed on Apr. 29, 2022, which is incorporated herein by reference in its entirety.

### SEQUENCE LISTING

This application contains a Sequence Listing that has been submitted electronically as an XML file named 44807-0425WO1_SL26.xml. The XML file, created on Apr. 27, 2023, is 25,912 bytes in size. The material in the XML file is hereby incorporated by reference in its entirety.

### TECHNICAL FIELD

The present disclosure relates to the field of biotechnology, and more specifically, to gene therapy to enhance mRNA expression in haploinsufficiency disorders.

### BACKGROUND

Haploinsufficiency occurs when one gene allele is inactivated and the amount of gene product expressed from the remaining active allele is insufficient for proper gene function. A number of disorders are associated with, or are caused by haploinsufficiency. In the treatment of haploinsufficiencies, targeting messenger RNA (mRNA) expression may offer a novel therapeutic window. Though changes in gene expression are commonly considered to reflect programmed transcriptional variability, it is a lesser-known fact that extensive regulation of messenger RNA expression also occurs during their translation. mRNA translation rate is a key feature defining post-transcriptional regulation. All transcripts are translated at unique rates and these rates can be controlled; dramatically impacting protein output per mRNA molecule. The cell achieves translational regulation through sequence and/or structural elements that recruit specific positive or negative acting factors to mRNAs.

Human mRNAs transmit genetic information from DNA to protein. Not only do mRNA transmit genetic information accurately, they also confer this information at the correct level. The amount of protein that comes from an mRNA is intrinsically timed. And the key feature that times how long an mRNA with make its protein is its 3′ polyadenosine tail.

Nearly all human mRNAs bear a long polyadenosine tail on their 3′ end. This tail has an average length of ~200 nt in humans. The poly(A) tail serves as a master regulator of gene expression in the cytoplasm. As long as an mRNA has a poly(A) tail, it will be translated. But once in the cytoplasm, an mRNA's poly(A) tail is subject to timed removal by a deadenylase enzyme complex. As soon as the tail is removed, the mRNA stops translating and is typically destroyed. Thus the poly(A) tail acts like a slow burning fuse, dictating how long a single mRNA will continue to make protein.

### SUMMARY

Provided herein are systems comprising: (a) an RNA binding moiety; and (b) a gRNA hybrid comprising (i) a complementary region that hybridizes with an mRNA of an active allele of a gene associated with a disorder associated with a decrease in the expression of a protein from the mRNA and (ii) a poly(A) tail; wherein the gRNA hybrid forms a complex with the RNA binding moiety.

In some embodiments, the RNA binding moiety is a Cas protein selected from the group consisting of Cas9, Cas12, Cas13, and Cas14. In some embodiments, the RNA binding moiety is Cas13b. In some embodiments, the RNA binding moiety is a catalytically inactive Cas protein.

In some embodiments, the poly(A) tail is located at the 5′ end of the gRNA hybrid. In some embodiments, the poly(A) tail is located at the 3′ end of the gRNA hybrid. In some embodiments, the poly(A) tail comprises about 30 nucleotides. In some embodiments, the poly(A) tail comprises about 50 nucleotides. In some embodiments, the poly(A) tail comprises about 75 nucleotides.

In some embodiments, the disorder is a haploinsufficiency disorder.

In some embodiments, the RNA binding moiety is encoded by a sequence that comprises or consists of SEQ ID NO: 1 or a sequence that has at least 80%, at least 85%, at least 90%, at least 95%, or at least 99% sequence identity to SEQ ID NO: 1. In some embodiments, the gRNA hybrid is encoded by a sequence comprising or consisting of SEQ ID NO: 2, SEQ ID NO: 3, or SEQ ID NO: 4.

Also provided herein are recombinant expression systems comprising: (a) a nucleic acid sequence encoding an RNA binding moiety; and (b) a nucleic acid sequence encoding a gRNA hybrid comprising (i) a complementary region that hybridizes with an mRNA of an active allele of a gene associated with a disorder associated with a decrease in the expression of a protein from the mRNA and (ii) a poly(A) tail; wherein the gRNA hybrid forms a complex with the RNA binding moiety.

In some embodiments, the RNA binding moiety is a Cas protein selected from the group consisting of Cas9, Cas12, Cas13, and Cas14. In some embodiments, the RNA binding moiety is Cas13b. In some embodiments, the RNA binding moiety is a catalytically inactive Cas protein.

In some embodiments, the poly(A) tail is located at the 5′ end of the gRNA hybrid. In some embodiments, the poly(A) tail is located at the 3′ end of the gRNA hybrid. In some embodiments, the poly(A) tail comprises about 30 nucleotides. In some embodiments, the poly(A) tail comprises about 50 nucleotides. In some embodiments, the poly(A) tail comprises about 75 nucleotides.

In some embodiments, the disorder is a haploinsufficiency disorder.

In some embodiments, the RNA binding moiety is encoded by a sequence that comprises or consists of SEQ ID NO: 1 or a sequence that has at least 80%, at least 85%, at least 90%, at least 95%, or at least 99% sequence identity to SEQ ID NO: 1. In some embodiments, the gRNA hybrid is encoded by a sequence comprising or consisting of SEQ ID NO: 2, SEQ ID NO: 3, or SEQ ID NO: 4.

Also provided herein are expression vectors comprising any one of the recombinant expression systems described herein. In some embodiments, the expression vector is a viral vector. In some embodiments, the viral vector is an adeno-associated viral vector (AAV), a lentiviral vector, or an adenoviral vector.

Also provided herein are cells comprising any one of the recombinant expression systems described herein or any one of the expression vectors described herein.

Also provided herein are methods of treating or preventing a haploinsufficiency disorder in a subject, the method comprising: administering any one of the recombinant expression systems described herein or any one of the expression vectors described herein.

In some embodiments, the haploinsufficiency disorder is selected from the group consisting from 5qsyndrome, Adams-Oliver syndrome 1, Adams-Oliver syndrome 3, Adams-Oliver syndrome 5, Adams-Oliver syndrome 6, Alagille syndrome 1, Autoimmune lymphoproliferative syndrome type IA, Autoimmune lymphoproliferative syndrome type V, Autosomal dominant deafness-2A, Brain malformations with or without urinary tract defects (BRMUTD), Carney complex type 1, CHARGE syndrome, Cleidocranial dysplasia, Currarino syndrome, Denys-Drash syndrome/Frasier syndrome, Developmental delay, intellectual disability, obesity, and dysmorphic features (DIDOD), DiGeorge syndrome (TBXI-associated), Dravet syndrome, Duane-radial raysyndrome, Ehlers-Danlos syndrome (classic-like), Ehlers-Danlos syndrome (vascular type), Feingold syndrome 1, Frontotemporal lobar degeneration with TDP43 inclusions (FTLD-TDP), GRN-related, GLUT I deficiency syndrome, Greig cephalopolysyndactyly syndrome, Hereditary hemorrhagic telangiectasia type 1, Holoprosencephaly 3, Holoprosencephaly 4, Holoprosencephaly 5, Holt-Oram syndrome, Hypoparathyroidism, sensorineural deafness, andrenal disease (HDR), Kleefstra syndrome 1, Klippel-Trenaunay syndrome (AAGF-related), Leri-Weill dyschondrosteosis, Marfan syndrome, Mental retardation and distinctive facial features with or without cardiac defects (MRFACD), Mental retardation, autosomal dominant 1, Mental retardation, autosomal dominant 19, Mental retardation, autosomal dominant 29, Nail-patella syndrome (NPS), Phelan-McDermid syndrome, Pitt-Hopkins syndrome, Primary pulmonary hypertension 1, Rett syndrome (congenital variant), Smith-Magenis syndrome (RAII associated), Sotos syndrome 1, Sotos syndrome 2, Stickler syndrome type I, Supravalvular aorticstenosis, SYNGAPI-related intellectual disability, Treacher Collins syndrome, Trichorhinophalangeal syndrome type I, Ulnar-mammary syndrome, van der Woude syndrome1, Waardenburg syndrome type 1, W aardenburg syndrome type 2A, and Waardenburg syndrometype 4C.

In some embodiments, the haploinsufficiency disorder is a CNS haploinsufficiency disorder. In some embodiments, the CNS haploinsufficiency disorder is selected from the group consisting of episodic ataxia, familial hemiplegia migraine, CDKL5 deficiency disorder, CHD2 myoclonic encephalopathy, familial focal epilepsy with variable loci, FOXG1 syndrome, benign familial neonatal seizures, Rett syndrome, Dravat syndrome, SCN2A-epileptic encephalopathy, SCN2A-developmental encephalopathy, SCN8A-epileptic encephalopathy, SC8A familial infantile epilepsy, early infantile epileptic encephalopathy, myoclonic-atonic epilepsy, early infantile epileptic encephalopathy, SYNGAP1-related intellectual disability, tuberous sclerosis, Lennox-Gastaut Syndrome, FoxG1 syndrome, KCNQ2-related epileptic encephalopathy, PCDH19-related epilepsy, SLC6A1-related myoclonic-astatic epilepsy, STXBP1-related epileptic encephalopathy, SYNGAP1 syndrome, and combinations thereof.

In some embodiments, the subject is a mammal. In some embodiments, the subject is a human.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 shows an exemplary schematic describing a therapeutic approach for the treatment of haploinsufficiency disorders, wherein the approach targets the normal copy of messenger RNA and restoring protein expression to normal amounts.

FIG. 2 shows the results of western blotting in SH-SY5Y cells co-transfected with Cas13 and Mecp2 guide RNA, where the results show significant increase in the level Mecp2 vs. control. Here, Cas13 combined with PAB protein and Mecp2-g6 without poly A tail was used as a control.

FIG. 3 shows the results of immunoblotting for the second biological repeat with more controls, where the results also confirmed the increased level of Mecp2 in 30 and 50 A tail guide RNA treated cells.

FIG. 4 shows results from an immunoblotting assay and its quantification for the third biological repeat, the results confirming the result of earlier experiments.

FIG. 5 shows results of qRT-PCR, the results also indicated about 20% increase in the level of Mecp2 RNA mostly in 50polyA guide RNA.

FIG. 6 shows a map of a dCas13b plasmid (pJC 1280).

FIG. 7 shows a map of a guide RNA (pJC 1276).

### DETAILED DESCRIPTION

The present disclosure is based on the discovery that targeting messenger RNA (mRNA) expression may offer a therapeutic method of modulating mRNA translation and/or treating haploinsufficiency disorders. mRNA translation rate is a key feature defining post-transcriptional regulation, where all transcripts are translated at unique rates and these rates can be controlled, dramatically impacting protein output per mRNA molecule. The cell achieves translational regulation through sequence and/or structural elements that recruit specific positive or negative acting factors to mRNAs.

The presently provided systems and methods can provide for disease modifying treatment for haploinsufficiency disorders by using a key positive acting mRNA regulator (e.g., the polyA tail) to bind and remain resident with the mRNA, thereby enhancing the wild-type (WT) mRNA's expression in a precise manner and restoring protein levels to normal.

In some embodiments, provided herein are systems that include (a) an RNA binding moiety; and (b) a gRNA comprising (i) a complementary region that hybridizes with an mRNA of an active allele of a gene associated with a disorder associated with a decrease in the expression of a protein from the mRNA and (ii) a poly(A) tail; wherein the gRNA forms a complex with the RNA binding moiety.

Also provided herein are recombinant expression systems that include (a) a nucleic acid sequence encoding an RNA binding moiety; and (b) a nucleic acid sequence encoding a gRNA comprising (i) a complementary region that hybridizes with an mRNA of an active allele of a gene associated with a disorder associated with a decrease in the expression of a protein from the mRNA and (ii) a poly(A) tail; wherein the gRNA forms a complex with the RNA binding moiety.

Various non-limiting aspects of these systems are described herein, and can be used in any combination without limitation. Additional aspects of various components of these systems are known in the art.

As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

As is known in the art, “affinity” is a measure of the tightness with a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

As used herein, the term “binding” typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts-including where interacting entities or moieties are studied in isolation or in the context of more complex systems (e.g., while covalently or otherwise associated with a carrier entity and/or in a biological system or cell).

As used herein, the term “engineered” refers to the aspect of having been manipulated by the hand of man. For example, a polypeptide is considered to be “engineered” when the polypeptide sequence manipulated by the hand of man. For example, in some embodiments of the present invention, an engineered polypeptide comprises a sequence that includes one or more amino acid mutations, deletions and/or insertions that have been introduced by the hand of man into a reference polypeptide sequence. In some embodiments, an engineered polypeptide includes a polypeptide that has been fused (i.e., covalently linked) to one or more additional polypeptides by the hand of man, to form a fusion polypeptide that would not naturally occur in vivo. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, derivatives and/or progeny of an engineered polypeptide or cell are typically still referred to as “engineered” even though the actual manipulation was performed on a prior entity.

As used herein, the term “pharmaceutical composition” refers to a composition in which an active agent is formulated together with one or more pharmaceutically acceptable carriers. In some embodiments, the composition is suitable for administration to a human or animal subject. In some embodiments, the active agent is present in unit dose amount appropriate for administration in a therapeutic regimen that shows a statistically significant probability of achieving a predetermined therapeutic effect when administered to a relevant population.

As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

As used herein, the term “subject” refers an organism, typically a mammal (e.g., a human). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Systems for Enhancing mRNA Translation

In some embodiments, provided herein are systems that include (a) an RNA binding moiety; and (b) a gRNA comprising (i) a complementary region that hybridizes with an mRNA of an active allele of a gene associated with a disorder associated with a decrease in the expression of a protein from the mRNA and (ii) a poly(A) tail; wherein the gRNA forms a complex with the RNA binding moiety.

Also provided herein are recombinant expression systems that include (a) a nucleic acid sequence encoding an RNA binding moiety; and (b) a nucleic acid sequence encoding a gRNA comprising (i) a complementary region that hybridizes with an mRNA of an active allele of a gene associated with a disorder associated with a decrease in the expression of a protein from the mRNA and (ii) a poly(A) tail; wherein the gRNA forms a complex with the RNA binding moiety.

In some embodiments, provided herein are expression vectors (e.g., a single vector or a plurality of vectors) including any one of the recombinant expression systems described herein. In some embodiments, the expression vector is a viral vector. In some embodiments, the viral vector is an adeno-associated viral vector (AAV), a lentiviral vector, or an adenoviral vector. In some embodiments, also provided herein are cells comprising any one of the recombinant expression systems or any one of the expression vectors described herein.

As used herein, an “RNA binding moiety” refers to a molecule or moiety capable of binding to an RNA (e.g., target RNA). In some embodiments, an RNA binding moiety can be fused to a protein (e.g., RNA binding protein). In some embodiments, an RNA binding moiety can include a reporter mRNA. In some embodiments, an RNA binding moiety can be attached to a protein through an artificial RNA-protein interaction. In some embodiments, an RNA binding moiety can include a MS2 bacteriophage coat protein (MCP). In some embodiments, an RNA binding moiety can include CRISPR components. For example, in some embodiments, CRISPR components can include, but are not limited to, a guide RNA and a CRISPR-associated endonuclease (Cas protein). In some embodiments, an RNA binding moiety can include a CRISPR-associated endonuclease (Cas protein). In some embodiments, a CRISPR-associated endonuclease (Cas protein) is engineered to reduce or deactivate the endonuclease activity.

### Cas Proteins

As used herein, a “Cas protein” refers to a CRISPR-associated protein which, in wild-type form, is an enzyme that uses CRISPR sequences as a guide to recognize and cleave specific nucleic acid strands that are complementary to the CRISPR sequence. The term “CRISPR” refers to a technique of sequence specific genetic manipulation relying on the clustered regularly interspaced short palindromic repeats (CRISPR) pathway. A CRISPR-associated protein (Cas protein) can associate with a CRISPR RNA sequence to bind to, and alter DNA or RNA target sequences. In some embodiments, a Cas protein can be a Cas9, a Cas12a, or a Cas13 nuclease (e.g., an engineered Cas9, Cas12a, or Cas13 nuclease) which targets RNA.

In some embodiments, the RNA binding moiety is a Cas protein or variant or mutant thereof. In some embodiments, the RNA binding moiety is Cas9 or a mutant or variant thereof. In some embodiments, the RNA binding moiety is a catalytically inactive Cas9 (e.g., a Cas9 with eliminated cleavage activity (dCas9)). In some embodiments, the RNA binding moiety is a Cas12 (e.g., Cas12a (Cpf1), LbCas12a or a mutant or variant thereof, or a mutant or variant with eliminated cleavage activity). In some embodiments, the RNA binding moiety is a Cas12b (e.g., AapCas12b, AacCas12b, or a mutant or variant with eliminated cleavage activity). In some embodiments, the RNA binding moiety is Cas13 or a mutant or variant thereof. See, e.g., Harrington et al., “Programmed DNA Destruction by Miniature CRISPR-Cas13 Enzymes,” Science 362 (6146): 839-42 (2018); Karvelis et al., “PAM Recognition by Miniature CRISPR-Cas12f Nucleases Triggers Programmable Double-Stranded DNA Target Cleavage,” Nucleic Acids Res 48 (9): 5016-23 (2020).

Type VI CRISPR-Cas systems contain the programmable single-effector RNA-guided RNases of the Cas13 family. See, e.g., Cox et al., “RNA Editing with CRISPR-Cas13,” Science 358 (6366): 1019-27 (2017). The Cas13 family contains at least four known subtypes, including Cas13a (formerly C2c2), Cas13b, Cas13c, and Cas13d. In some embodiments, the RNA binding moiety is a RNA-targeting Cas13, e.g., Cas13a, Cas13b, Cas13c, or Cas13d. In some embodiments, the RNA binding moiety is Cas13b. See, e.g., Smargon et al. (2017), “Cas13b Is a Type VI-B CRISPR-Associated RNA-Guided RNase Differentially Regulated by Accessory Proteins Csx27 and Csx28,” Molecular Cell 65, 618-630; Smargon et al., “RNA-Targeting CRISPR Systems from Metagenomic Discovery to Transcriptome Engineering,” Nat Cell Biol 22 (2): 143-50 (2020). In some embodiments, the RNA binding moiety is a catalytically inactive RNA binding moiety (e.g., a Cas13 with eliminated cleavage activity (dCas13), dCas13b). In some embodiments, the Cas13 is a Cas13bt. See, e.g., Kannan et al., “Compact RNA Editors with Small Cas13 Proteins,” Nature Biotechnology 18:499-560 (2021). In some embodiments, the Cas13 is a catalytically inactive Cas13bt (dCas13bt). In some embodiments, the Cas13 is a Cas13bt with mutations corresponding to H133A and H1058 of dCas13b.

In some embodiments, the RNA binding moiety is a Cas protein selected from the group consisting of Cas9, Cas12, Cas13, and Cas14. In some embodiments, the RNA binding moiety is Cas13b. In some embodiments, the RNA binding moiety is a catalytically inactive Cas protein.

In some embodiments, the RNA binding moiety is encoded by a sequence that comprises or consists of SEQ ID NO: 1 or a sequence that has at least 80%, at least 85%, at least 90%, at least 95%, or at least 99% sequence identity to SEQ ID NO: 1.

dCas13b plasmid
SEQ ID NO: 1
CTTTAATAGTGGACTCTTGTTCCAAACTGGAACAACACTCAACCCTATCTCGGTCTAT
TCTTTTGATTTATAAGGGATTTTGCCGATTTCGGCCTATTGGTTAAAAAATGAGCTGA
TTTAACAAAAATTTAACGCGAATTAATTCTGTGGAATGTGTGTCAGTTAGGGTGTGG
AAAGTCCCCAGGCTCCCCAGCAGGCAGAAGTATGCAAAGCATGCATCTCAATTAGT
CAGCAACCAGGTGTGGAAAGTCCCCAGGCTCCCCAGCAGGCAGAAGTATGCAAAGC
ATGCATCTCAATTAGTCAGCAACCATAGTCCCGCCCCTAACTCCGCCCATCCCGCCC
CTAACTCCGCCCAGTTCCGCCCATTCTCCGCCCCATGGCTGACTAATTTTTTTTATTT
ATGCAGAGGCCGAGGCCGCCTCTGCCTCTGAGCTATTCCAGAAGTAGTGAGGAGGC
TTTTTTGGAGGCCTAGGCTTTTGCAAAAAGCTCCCGGGAGCTTGTATATCCATTTTCGGATC
TGATCAAGAGACAGGATGAGGATCGTTTCGCATGATTGAACAAGATGGATTG
CACGCAGGTTCTCCGGCCGCTTGGGTGGAGAGGCTATTCGGCTATGACTGGGCACAA
CAGACAATCGGCTGCTCTGATGCCGCCGTGTTCCGGCTGTCAGCGCAGGGGCGCCCG
GTTCTTTTTGTCAAGACCGACCTGTCCGGTGCCCTGAATGAACTGCAGGACGAGGCA
GCGCGGCTATCGTGGCTGGCCACGACGGGCGTTCCTTGCGCAGCTGTGCTCGACGTT
GTCACTGAAGCGGGAAGGGACTGGCTGCTATTGGGCGAAGTGCCGGGGCAGGATCT
CCTGTCATCTCACCTTGCTCCTGCCGAGAAAGTATCCATCATGGCTGATGCAATGCG
GCGGCTGCATACGCTTGATCCGGCTACCTGCCCATTCGACCACCAAGCGAAACATCG
CATCGAGCGAGCACGTACTCGGATGGAAGCCGGTCTTGTCGATCAGGATGATCTGG
ACGAAGAGCATCAGGGGCTCGCGCCAGCCGAACTGTTCGCCAGGCTCAAGGCGCGC
ATGCCCGACGGCGAGGATCTCGTCGTGACCCATGGCGATGCCTGCTTGCCGAATATC
ATGGTGGAAAATGGCCGCTTTTCTGGATTCATCGACTGTGGCCGGCTGGGTGTGGCG
GACCGCTATCAGGACATAGCGTTGGCTACCCGTGATATTGCTGAAGAGCTTGGCGGC
GAATGGGCTGACCGCTTCCTCGTGCTTTACGGTATCGCCGCTCCCGATTCGCAGCGC
ATCGCCTTCTATCGCCTTCTTGACGAGTTCTTCTGAGCGGGACTCTGGGGTTCGAAAT
GACCGACCAAGCGACGCCCAACCTGCCATCACGAGATTTCGATTCCACCGCCGCCTT
CTATGAAAGGTTGGGCTTCGGAATCGTTTTCCGGGACGCCGGCTGGATGATCCTCCA
GCGCGGGGATCTCATGCTGGAGTTCTTCGCCCACCCCAACTTGTTTATTGCAGCTTAT
AATGGTTACAAATAAAGCAATAGCATCACAAATTTCACAAATAAAGCATTTTTTTCA
CTGCATTCTAGTTGTGGTTTGTCCAAACTCATCAATGTATCTTATCATGTCTGTATACCGTCG
ACCTCTAGCTAGAGCTTGGCGTAATCATGGTCATAGCTGTTTCCTGTGTGAA
ATTGTTATCCGCTCACAATTCCACACAACATACGAGCCGGAAGCATAAAGTGTAAA
GCCTGGGGTGCCTAATGAGTGAGCTAACTCACATTAATTGCGTTGCGCTCACTGCCC
GCTTTCCAGTCGGGAAACCTGTCGTGCCAGCTGCATTAATGAATCGGCCAACGCGCG
GGGAGAGGCGGTTTGCGTATTGGGCGCTCTTCCGCTTCCTCGCTCACTGACTCGCTG
CGCTCGGTCGTTCGGCTGCGGCGAGCGGTATCAGCTCACTCAAAGGCGGTAATACG
GTTATCCACAGAATCAGGGGATAACGCAGGAAAGAACATGTGAGCAAAAGGCCAG
CAAAAGGCCAGGAACCGTAAAAAGGCCGCGTTGCTGGCGTTTTTCCATAGGCTCCG
CCCCCCTGACGAGCATCACAAAAATCGACGCTCAAGTCAGAGGTGGCGAAACCCGA
CAGGACTATAAAGATACCAGGCGTTTCCCCCTGGAAGCTCCCTCGTGCGCTCTCCTG
TTCCGACCCTGCCGCTTACCGGATACCTGTCCGCCTTTCTCCCTTCGGGAAGCGTGGC
GCTTTCTCATAGCTCACGCTGTAGGTATCTCAGTTCGGTGTAGGTCGTTCGCTCCAAGCTGGG
CTGTGTGCACGAACCCCCCGTTCAGCCCGACCGCTGCGCCTTATCCGGTAAC
TATCGTCTTGAGTCCAACCCGGTAAGACACGACTTATCGCCACTGGCAGCAGCCACT
GGTAACAGGATTAGCAGAGCGAGGTATGTAGGCGGTGCTACAGAGTTCTTGAAGTG
GTGGCCTAACTACGGCTACACTAGAAGAACAGTATTTGGTATCTGCGCTCTGCTGAA
GCCAGTTACCTTCGGAAAAAGAGTTGGTAGCTCTTGATCCGGCAAACAAACCACCG
CTGGTAGCGGTGGTTTTTTTGTTTGCAAGCAGCAGATTACGCGCAGAAAAAAAGGAT
CTCAAGAAGATCCTTTGATCTTTTCTACGGGGTCTGACGCTCAGTGGAACGAAAACT
CACGTTAAGGGATTTTGGTCATGAGATTATCAAAAAGGATCTTCACCTAGATCCTTT
TAAATTAAAAATGAAGTTTTAAATCAATCTAAAGTATATATGAGTAAACTTGGTCTG
ACAGTTACCAATGCTTAATCAGTGAGGCACCTATCTCAGCGATCTGTCTATTTCGTTC
ATCCATAGTTGCCTGACTCCCCGTCGTGTAGATAACTACGATACGGGAGGGCTTACC
ATCTGGCCCCAGTGCTGCAATGATACCGCGAGACCCACGCTCACCGGCTCCAGATTT
ATCAGCAATAAACCAGCCAGCCGGAAGGGCCGAGCGCAGAAGTGGTCCTGCAACTT
TATCCGCCTCCATCCAGTCTATTAATTGTTGCCGGGAAGCTAGAGTAAGTAGTTCGC
CAGTTAATAGTTTGCGCAACGTTGTTGCCATTGCTACAGGCATCGTGGTGTCACGCT
CGTCGTTTGGTATGGCTTCATTCAGCTCCGGTTCCCAACGATCAAGGCGAGTTACAT
GATCCCCCATGTTGTGCAAAAAAGCGGTTAGCTCCTTCGGTCCTCCGATCGTTGTCA
GAAGTAAGTTGGCCGCAGTGTTATCACTCATGGTTATGGCAGCACTGCATAATTCTC
TTACTGTCATGCCATCCGTAAGATGCTTTTCTGTGACTGGTGAGTACTCAACCAAGTC
ATTCTGAGAATAGTGTATGCGGCGACCGAGTTGCTCTTGCCCGGCGTCAATACGGGA
TAATACCGCGCCACATAGCAGAACTTTAAAAGTGCTCATCATTGGAAAACGTTCTTC
GGGGCGAAAACTCTCAAGGATCTTACCGCTGTTGAGATCCAGTTCGATGTAACCCAC
TCGTGCACCCAACTGATCTTCAGCATCTTTTACTTTCACCAGCGTTTCTGGGTGAGCAAAAAC
AGGAAGGCAAAATGCCGCAAAAAAGGGAATAAGGGCGACACGGAAATGTT
GAATACTCATACTCTTCCTTTTTCAATATTATTGAAGCATTTATCAGGGTTATTGTCT
CATGAGCGGATACATATTTGAATGTATTTAGAAAAATAAACAAATAGGGGTTCCGC
GCACATTTCCCCGAAAAGTGCCACCTGACGTCGACGGATCGGGAGATCTCCCGATCC
CCTATGGTGCACTCTCAGTACAATCTGCTCTGATGCCGCATAGTTAAGCCAGTATCT
GCTCCCTGCTTGTGTGTTGGAGGTCGCTGAGTAGTGCGCGAGCAAAATTTAAGCTAC
AACAAGGCAAGGCTTGACCGACAATTGCATGAAGAATCTGCTTAGGGTTAGGCGTT
TTGCGCTGCTTCGCGATGTACGGGCCAGATATACGCGTTGACATTGATTATTGACTA
GTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCC
CATTGACGTCAATAATGACGTATGTTCCCATAGTAACGCCAATAGGGACTTTCCATT
GACGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTGGC
ATTATGCCCAGTACATGACCTTATGGGACTTTCCTACTTGGCAGTACATCTACGTATT
AGTCATCGCTATTACCATGGTGATGCGGTTTTGGCAGTACATCAATGGGCGTGGATA
GCGGTTTGACTCACGGGGATTTCCAAGTCTCCACCCCATTGACGTCAATGGGAGTTT
GTTTTGGCACCAAAATCAACGGGACTTTCCAAAATGTCGTAACAACTCCGCCCCATT
GACGCAAATGGGCGGTAGGCGTGTACGGTGGGAGGTCTATATAAGCAGAGCTCTCT
GGCTAACTAGAGAACCCACTGCTTACTGGCTTATCGAAATTAATACGACTCACTATA
GGGAGACCCAAGCTGGCTAGCGTTTAAACTTAAGCTTGCCACCATGAACATCCCCGC
TCTGGTGGAAAACCAGAAGAAGTACTTTGGCACCTACAGCGTGATGGCCATGCTGA
ACGCTCAGACCGTGCTGGACCACATCCAGAAGGTGGCCGATATTGAGGGCGAGCAG
AACGAGAACAACGAGAATCTGTGGTTTCACCCCGTGATGAGCCACCTGTACAACGC
CAAGAACGGCTACGACAAGCAGCCCGAGAAAACCATGTTCATCATCGAGCGGCTGC
AGAGCTACTTCCCATTCCTGAAGATCATGGCCGAGAACCAGAGAGAGTACAGCAAC
GGCAAGTACAAGCAGAACCGCGTGGAAGTGAACAGCAACGACATCTTCGAGGTGCT
GAAGCGCGCCTTCGGCGTGCTGAAGATGTACAGGGACCTGACCAACGCATACAAGA
CCTACGAGGAAAAGCTGAACGACGGCTGCGAGTTCCTGACCAGCACAGAGCAACCT
CTGAGCGGCATGATCAACAACTACTACACAGTGGCCCTGCGGAACATGAACGAGAG
ATACGGCTACAAGACAGAGGACCTGGCCTTCATCCAGGACAAGCGGTTCAAGTTCG
TGAAGGACGCCTACGGCAAGAAAAAGTCCCAAGTGAATACCGGATTCTTCCTGAGC
CTGCAGGACTACAACGGCGACACACAGAAGAAGCTGCACCTGAGCGGAGTGGGAA
TCGCCCTGCTGATCTGCCTGTTCCTGGACAAGCAGTACATCAACATCTTTCTGAGCA
GGCTGCCCATCTTCTCCAGCTACAATGCCCAGAGCGAGGAACGGCGGATCATCATC
AGATCCTTCGGCATCAACAGCATCAAGCTGCCCAAGGACCGcATCCACAGCGAGAA
GTCCAACAAGAGCGTGGCCATGGATATGCTCAACGAAGTGAAGCGGTGCCCCGACG
AGCTGTTCACAACACTGTCTGCCGAGAAGCAGTCCCGGTTCAGAATCATCAGCGAC
GACCACAATGAAGTGCTGATGAAGCGGAGCAGCGACAGATTCGTGCCTCTGCTGCT
GCAGTATATCGATTACGGCAAGCTGTTCGACCACATCAGGTTCCACGTGAACATGGG
CAAGCTGAGATACCTGCTGAAGGCCGACAAGACCTGCATCGACGGCCAGACCAGAG
TCAGAGTGATCGAGCAGCCCCTGAACGGCTTCGGCAGACTGGAAGAGGCCGAGACA
ATGCGGAAGCAAGAGAACGGCACCTTCGGCAACAGCGGCATCCGGATCAGAGACTT
CGAGAACATGAAGCGGGACGACGCCAATCCTGCCAACTATCCCTACATCGTGGACA
CCTACACACACTACATCCTGGAAAACAACAAGGTCGAGATGTTTATCAACGACAAA
GAGGACAGCGCCCCACTGCTGCCCGTGATCGAGGATGATAGATACGTGGTCAAGAC
AATCCCCAGCTGCCGGATGAGCACCCTGGAAATTCCAGCCATGGCCTTCCACATGTT
TCTGTTCGGCAGCAAGAAAACCGAGAAGCTGATCGTGGACGTGCACAACCGGTACA
AGAGACTGTTCCAGGCCATGCAGAAAGAAGAAGTGACCGCCGAGAATATCGCCAGC
TTCGGAATCGCCGAGAGCGACCTGCCTCAGAAGATCCTGGATCTGATCAGCGGCAA
TGCCCACGGCAAGGATGTGGACGCCTTCATCAGACTGACCGTGGACGACATGCTGA
CCGACACCGAGCGGAGAATCAAGAGATTCAAGGACGACCGGAAGTCCATTCGGAGC
GCCGACAACAAGATGGGAAAGAGAGGCTTCAAGCAGATCTCCACAGGCAAGCTGG
CCGACTTCCTGGCCAAGGACATCGTGCTGTTTCAGCCCAGCGTGAACGATGGCGAG
AACAAGATCACCGGCCTGAACTACCGGATCATGCAGAGCGCCATTGCCGTGTACGA
TAGCGGCGACGATTACGAGGCCAAGCAGCAGTTCAAGCTGATGTTCGAGAAGGCCC
GGCTGATCGGCAAGGGCACAACAGAGCCTCATCCATTTCTGTACAAGGTGTTCGCCC
GCAGCATCCCCGCCAATGCCGTCGAGTTCTACGAGCGCTACCTGATCGAGCGGAAG
TTCTACCTGACCGGCCTGTCCAACGAGATCAAGAAAGGCAACAGAGTGGATGTGCC
CTTCATCCGGCGGGACCAGAACAAGTGGAAAACACCCGCCATGAAGACCCTGGGCA
GAATCTACAGCGAGGATCTGCCCGTGGAACTGCCCAGACAGATGTTCGACAATGAG
ATCAAGTCCCACCTGAAGTCCCTGCCACAGATGGAAGGCATCGACTTCAACAATGC
CAACGTGACCTATCTGATCGCCGAGTACATGAAGAGAGTGCTGGACGACGACTTCC
AGACCTTCTACCAGTGGAACCGCAACTACCGGTACATGGACATGCTTAAGGGCGAG
TACGACAGAAAGGGCTCCCTGCAGCACTGCTTCACCAGCGTGGAAGAGAGAGAAGG
CCTCTGGAAAGAGCGGGCCTCCAGAACAGAGCGGTACAGAAAGCAGGCCAGCAAC
AAGATCCGCAGCAACCGGCAGATGAGAAACGCCAGCAGCGAAGAGATCGAGACAA
TCCTGGATAAGCGGCTGAGCAACAGCCGGAACGAGTACCAGAAAAGCGAGAAAGT
GATCCGGCGCTACAGAGTGCAGGATGCCCTGCTGTTTCTGCTGGCCAAAAAGACCCT
GACCGAACTGGCCGATTTCGACGGCGAGAGGTTCAAACTGAAAGAAATCATGCCCG
ACGCCGAGAAGGGAATCCTGAGCGAGATCATGCCCATGAGCTTCACCTTCGAGAAA
GGCGGCAAGAAGTACACCATCACCAGCGAGGGCATGAAGCTGAAGAACTACGGCG
ACTTCTTTGTGCTGGCTAGCGACAAGAGGATCGGCAACCTGCTGGAACTCGTGGGCA
GCGACATCGTGTCCAAAGAGGATATCATGGAAGAGTTCAACAAATACGACCAGTGC
AGGCCCGAGATCAGCTCCATCGTGTTCAACCTGGAAAAGTGGGCCTTCGACACATA
CCCCGAGCTGTCTGCCAGAGTGGACCGGGAAGAGAAGGTGGACTTCAAGAGCATCC
TGAAAATCCTGCTGAACAACAAGAACATCAACAAAGAGCAGAGCGACATCCTGCGG
AAGATCCGGAACGCCTTCGATGCAAACAATTACCCCGACAAAGGCGTGGTGGAAAT
CAAGGCCCTGCCTGAGATCGCCATGAGCATCAAGAAGGCCTTTGGGGAGTACGCCA
TCATGAAGGGAAGCCTGCAGGCGGCCGCTCGAGCCTAGAGGGCCCGTTTAAACCCG
CTGATCAGCCTCGACTGTGCCTTCTAGTTGCCAGCCATCTGTTGTTTGCCCCTCCCCC
GTGCCTTCCTTGACCCTGGAAGGTGCCACTCCCACTGTCCTTTCCTAATAAAATGAG
GAAATTGCATCGCATTGTCTGAGTAGGTGTCATTCTATTCTGGGGGGTGGGGTGGGG
CAGGACAGCAAGGGGGAGGATTGGGAAGACAATAGCAGGCATGCTGGGGATGCGG
TGGGCTCTATGGCTTCTGAGGCGGAAAGAACCAGCTGGGGCTCTAGGGGGTATCCC
CACGCGCCCTGTAGCGGCGCATTAAGCGCGGCGGGTGTGGTGGTTACGCGCAGCGT
GACCGCTACACTTGCCAGCGCCCTAGCGCCCGCTCCTTTCGCTTTCTTCCCTTCCTTT
CTCGCCACGTTCGCCGGCTTTCCCCGTCAAGCTCTAAATCGGGGGCTCCCTTTAGGG
TTCCGATTTAGTGCTTTACGGCACCTCGACCCCAAAAAACTTGATTAGGGTGATGGT
TCACGTAGTGGGCCATCGCCCTGATAGACGGTTTTTCGCCCTTTGACGTTGGAGTCCACGTT

### Guide RNA

As used herein, the term “gRNA” or “guide RNA” refers to the guide RNA sequences used to target specific genes for correction employing the CRISPR technique. Techniques of designing gRNAs and donor therapeutic polynucleotides for target specificity are well known in the art. See, e.g., Doench, J., et al. Nature biotechnology 2014; 32 (12): 1262-7; Graham, D., et al. Genome Biol. 2015; 16:260. In some embodiments, a gRNA can comprise tracrRNA (transactivating RNA), which binds to Cas9. In some embodiments, a gRNA can include crRNA (CRISPR RNA), comprising complimentary nucleotides to the tracrRNA, into a single RNA construct. Exemplary methods of employing the CRISPR technique are described in WO 2017/091630, which is incorporated by reference in its entirety.

In some embodiments, the guide RNA can recognize a target RNA, for example, by hybridizing to the target RNA. In some embodiments, the guide RNA comprises a sequence that is complementary to the target RNA. In some embodiments, the guide RNA comprises a sequence that is complementary to a sequence in the 3′ untranslated region (3′ UTR) of the target RNA. In some embodiments, the gRNA can include one or more modified nucleotides. In some embodiments, the gRNA has a length that is about 10 nt (e.g., about 20 nt, about 30 nt, about 40 nt, about 50 nt, about 60 nt, about 70 nt, about 80 nt, about 90 nt, about 100 nt, about 120 nt, about 140 nt, about 160 nt, about 180 nt, about 200 nt, about 300 nt, about 400 nt, about 500 nt, about 600 nt, about 700 nt, about 800 nt, about 900 nt, about 1000 nt, or about 2000 nt).

In some embodiments, a guide RNA can recognize any of a variety of RNA targets. For example, a target RNA can be messenger RNA (mRNA), ribosomal RNA (rRNA), signal recognition particle RNA (SRP RNA), transfer RNA (tRNA), small nuclear RNA (snRNA), small nucleolar RNA (snoRNA), antisense RNA (aRNA), long noncoding RNA (lncRNA), microRNA (miRNA), piwi-interacting RNA (piRNA), small interfering RNA (siRNA), short hairpin RNA (shRNA), retrotransposon RNA, viral genome RNA, or viral noncoding RNA. In some embodiments, a target RNA can be an RNA involved in pathogenesis of conditions such as cancers, neurodegeneration, cutaneous conditions, endocrine conditions, intestinal diseases, infectious conditions, neurological conditions, liver diseases, heart disorders, haploinsufficiency disorders, or autoimmune diseases. In some embodiments, a target RNA can be a therapeutic target for conditions such as cancers, neurodegeneration, cutaneous conditions, endocrine conditions, intestinal diseases, infectious conditions, neurological conditions, liver diseases, heart disorders, haploinsufficiency disorders, or autoimmune diseases.

In some embodiments, the guide RNA (e.g., of a gRNA hybrid) comprises a sequence that is complementary to a sequence in the 3′ untranslated region (3′ UTR) of the target RNA. In some embodiments, the gRNA comprises a sequence that is complementary to a sequence in the 3′ untranslated region (3′ UTR) of the target RNA, wherein the target RNA is MeCP2. In some embodiments, the gRNA comprises SEQ ID NO: 8. In some embodiments, the gRNA hybrid sequence is encoded by a vector. In some embodiments, the vector comprises a sequence of one of SEQ ID NOs: 2, 3, or 4 and its reverse complement. The sequences of the gRNA and poly(A) tail are indicated by capital letters.

```
guide RNA vector (pJC1276)
SEQ ID NO: 2
agtggctgctgccagtggcgataagtcgtgtcttaccgggttggactcaagacgatagttaccggataaggcgcagcggtcgggctgaac
ggggggttcgtgcacacagcccagcttggagcgaacgacctacaccgaactgagatacctacagcgtgagctatgagaaagcgccacg
cttcccgaagggagaaaggcggacaggtatccggtaagcggcagggtcggaacaggagagcgcacgagggagcttccagggggaaa
cgcctggtatctttatagtcctgtcgggtttcgccacctctgacttgagcgtcgatttttgtgatgctcgtcaggggggcggagcctatggaaaa
acgccagcaacgcggcctttttacggttcctggccttttgctggccttttgctcacatgttctttcctgcgttatcccctgattctgtggataaccgt
attaccgcctttgagtgagctgataccgctcgccgcagccgaacgaccgagcgcagcgagtcagtgagcgaggaagcggaagagcgcc
caatacgcaaaccgcctctccccgcgcgttggccgattcattaatgcagctggcacgacaggtttcccgactggaaagcgggcagtgagc
gcaacgcaattaatgtgagttagctcactcattaggcaccccaggctttacactttatgcttccggctcgtatgttgtgtggaattgtgagcggat
aacaatttcacgcaggaaacagctatgaccatgattacgccagagggcctatttcccatgattccttcatatttgcatatacgatacaaggctgt
tagagagataattggaattaatttgactgtaaacacaaagatattagtacaaaatacgtgacgtagaaagtaataatttcttgggtagtttgcagt
tttaaaattatgttttaaaatggactatcatatgcttaccgtaacttgaaagtatttcgatttcttggctttatatatcttgtggaaaggacgaaacacc
atcttgtggaaaggacgaaacaccgAAAAAAAAAAAAAAAAAAAAAAAAAAAAAAGCTAAAAAT
GTATATGCCCAAAGcaagttgtggaaggtccagttttatgtcttcctgggacgaagacaagttgtggaaggtccagttttgaggg
gctattacaacttttttggtaccgagctcgaattcactggccgtcgttttacaacgtcgtgactgggaaaaccctggcgttacccaacttaatcg
ccttgcagcacatccccctttcgccagctggcgtaatagcgaagaggcccgcaccgatcgcccttcccaacagttgcgcagcctgaatggc
gaatggcgcctgatgcggtattttctccttacgcatctgtgcggtatttcacaccgcatatggtgcactctcagtacaatctgctctgatgccgc
atagttaagccagccccgacacccgccaacacccgctgacgcgccctgacgggcttgtctgctcccggcatccgcttacagacaagctgt
gaccgtctccgggagctgcatgtgtcagaggttttcaccgtcatcaccgaaacgcgcgagacgaaagggcctcgtgatacgcctatttttat
aggttaatgtcatgataataatggtttcttagacgtcaggtggcacttttcggggaaatgtgcgcggaacccctatttgtttatttttctaaatacat
tcaaatatgtatccgctcatgagacaataaccctgataaatgcttcaataatattgaaaaaggaagagtatgagtattcaacatttccgtgtcgc
ccttattcccttttttgcggcattttgccttcctgtttttgctcacccagaaacgctggtgaaagtaaaagatgctgaagatcagttgggtgcacga
gtgggttacatcgaactggatctcaacagcggtaagatccttgagagttttcgccccgaagaacgttttccaatgatgagcacttttaaagttct
gctatgtggcgcggtattatcccgtattgacgccgggcaagagcaactcggtcgccgcatacactattctcagaatgacttggttgagtactc
accagtcacagaaaagcatcttacggatggcatgacagtaagagaattatgcagtgctgccataaccatgagtgataacactgcggccaac
ttacttctgacaacgatcggaggaccgaaggagctaaccgcttttttgcacaacatgggggatcatgtaactcgccttgatcgttgggaaccg
gagctgaatgaagccataccaaacgacgagcgtgacaccacgatgcctgtagcaatggcaacaacgttgcgcaaactattaactggcgaa
ctacttactctagcttcccggcaacaattaatagactggatggaggcggataaagttgcaggaccacttctgcgctcggcccttccggctggc
tggtttattgctgataaatctggagccggtgagcgtgggtctcgcggtatcattgcagcactggggccagatggtaagccctcccgtatcgta
gttatctacacgacggggagtcaggcaactatggatgaacgaaatagacagatcgctgagataggtgcctcactgattaagcattggtaact
gtcagaccaagtttactcatatatactttagattgatttaaaacttcatttttaatttaaaaggatctaggtgaagatcctttttgataatctcatgacc
aaaatcccttaacgtgagttttcgttccactgagcgtcagaccccgtagaaaagatcaaaggatcttcttgagatcctttttttctgcgcgtaatct
gctgcttgcaaacaaaaaaaccaccgctaccagcggtggtttgtttgccggatcaagagctaccaactctttttccgaaggtaactggcttca
gcagagcgcagataccaaatactgttcttctagtgtagccgtagttaggccaccacttcaagaactctgtagcaccgcctacatacctcgctct
gctaatcctgttacc
guide RNA vector (pJc 1277)
SEQ ID NO: 3
agtggctgctgccagtggcgataagtcgtgtcttaccgggttggactcaagacgatagttaccggataaggcgcagcggtcgggctgaac
ggggggttcgtgcacacagcccagcttggagcgaacgacctacaccgaactgagatacctacagcgtgagctatgagaaagcgccacg
cttcccgaagggagaaaggcggacaggtatccggtaagcggcagggtcggaacaggagagcgcacgagggagcttccagggggaaa
cgcctggtatctttatagtcctgtcgggtttcgccacctctgacttgagcgtcgatttttgtgatgctcgtcaggggggcggagcctatggaaaa
acgccagcaacgcggcctttttacggttcctggccttttgctggccttttgctcacatgttctttcctgcgttatcccctgattctgtggataaccgt
attaccgcctttgagtgagctgataccgctcgccgcagccgaacgaccgagcgcagcgagtcagtgagcgaggaagcggaagagcgcc
caatacgcaaaccgcctctccccgcgcgttggccgattcattaatgcagctggcacgacaggtttcccgactggaaagcgggcagtgagc
gcaacgcaattaatgtgagttagctcactcattaggcaccccaggctttacactttatgcttccggctcgtatgttgtgtggaattgtgagcggat
aacaatttcacgcaggaaacagctatgaccatgattacgccagagggcctatttcccatgattccttcatatttgcatatacgatacaaggctgt
tagagagataattggaattaatttgactgtaaacacaaagatattagtacaaaatacgtgacgtagaaagtaataatttcttgggtagtttgcagt
tttaaaattatgttttaaaatggactatcatatgcttaccgtaacttgaaagtatttcgatttcttggctttatatatcttgtggaaaggacgaaacacc
AAAAAAAAAAAAAAAAAAAAAAAAAAAAAAAAAAAAAAAAAAAAAAAAAAGCTA
AAAATGTATATGCCCAAAGcaagttgtggaaggtccagttttatgtcttcctgggacgaagacaagttgtggaaggtccag
ttttgaggggctattacaacttttttggtaccgagctcgaattcactggccgtcgttttacaacgtcgtgactgggaaaaccctggcgttaccca
acttaatcgccttgcagcacatccccctttcgccagctggcgtaatagcgaagaggcccgcaccgatcgcccttcccaacagttgcgcagc
ctgaatggcgaatggcgcctgatgcggtattttctccttacgcatctgtgcggtatttcacaccgcatatggtgcactctcagtacaatctgctct
gatgccgcatagttaagccagccccgacacccgccaacacccgctgacgcgccctgacgggcttgtctgctcccggcatccgcttacaga
caagctgtgaccgtctccgggagctgcatgtgtcagaggttttcaccgtcatcaccgaaacgcgcgagacgaaagggcctcgtgatacgc
ctatttttataggttaatgtcatgataataatggtttcttagacgtcaggtggcacttttcggggaaatgtgcgcggaacccctatttgtttatttttct
aaatacattcaaatatgtatccgctcatgagacaataaccctgataaatgcttcaataatattgaaaaaggaagagtatgagtattcaacatttcc
gtgtcgcccttattcccttttttgcggcattttgccttcctgtttttgctcacccagaaacgctggtgaaagtaaaagatgctgaagatcagttggg
tgcacgagtgggttacatcgaactggatctcaacagcggtaagatccttgagagttttcgccccgaagaacgttttccaatgatgagcactttt
aaagttctgctatgtggcgcggtattatcccgtattgacgccgggcaagagcaactcggtcgccgcatacactattctcagaatgacttggttg
agtactcaccagtcacagaaaagcatcttacggatggcatgacagtaagagaattatgcagtgctgccataaccatgagtgataacactgcg
gccaacttacttctgacaacgatcggaggaccgaaggagctaaccgcttttttgcacaacatgggggatcatgtaactcgccttgatcgttgg
gaaccggagctgaatgaagccataccaaacgacgagcgtgacaccacgatgcctgtagcaatggcaacaacgttgcgcaaactattaact
ggcgaactacttactctagcttcccggcaacaattaatagactggatggaggcggataaagttgcaggaccacttctgcgctcggcccttcc
ggctggctggtttattgctgataaatctggagccggtgagcgtgggtctcgcggtatcattgcagcactggggccagatggtaagccctccc
gtatcgtagttatctacacgacggggagtcaggcaactatggatgaacgaaatagacagatcgctgagataggtgcctcactgattaagcat
tggtaactgtcagaccaagtttactcatatatactttagattgatttaaaacttcatttttaatttaaaaggatctaggtgaagatcctttttgataatct
catgaccaaaatcccttaacgtgagttttcgttccactgagcgtcagaccccgtagaaaagatcaaaggatcttcttgagatcctttttttctgcg
cgtaatctgctgcttgcaaacaaaaaaaccaccgctaccagcggtggtttgtttgccggatcaagagctaccaactctttttccgaaggtaact
ggcttcagcagagcgcagataccaaatactgttcttctagtgtagccgtagttaggccaccacttcaagaactctgtagcaccgcctacatac
ctcgctctgctaatcctgttacc
  guide RNA vector (pJc 1278)
SEQ ID NO: 4
agtggctgctgccagtggcgataagtcgtgtcttaccgggttggactcaagacgatagttaccggataaggcgcagcggtcgggctgaac
ggggggttcgtgcacacagcccagcttggagcgaacgacctacaccgaactgagatacctacagcgtgagctatgagaaagcgccacg
cttcccgaagggagaaaggcggacaggtatccggtaagcggcagggtcggaacaggagagcgcacgagggagcttccagggggaaa
cgcctggtatctttatagtcctgtcgggtttcgccacctctgacttgagcgtcgatttttgtgatgctcgtcaggggggcggagcctatggaaaa
acgccagcaacgcggcctttttacggttcctggccttttgctggccttttgctcacatgttctttcctgcgttatcccctgattctgtggataaccgt
attaccgcctttgagtgagctgataccgctcgccgcagccgaacgaccgagcgcagcgagtcagtgagcgaggaagcggaagagcgcc
caatacgcaaaccgcctctccccgcgcgttggccgattcattaatgcagctggcacgacaggtttcccgactggaaagcgggcagtgagc
gcaacgcaattaatgtgagttagctcactcattaggcaccccaggctttacactttatgcttccggctcgtatgttgtgtggaattgtgagcggat
aacaatttcacgcaggaaacagctatgaccatgattacgccagagggcctatttcccatgattccttcatatttgcatatacgatacaaggctgt
tagagagataattggaattaatttgactgtaaacacaaagatattagtacaaaatacgtgacgtagaaagtaataatttcttgggtagtttgcagt
tttaaaattatgttttaaaatggactatcatatgcttaccgtaacttgaaagtatttcgatttcttggctttatatatcttgtggaaaggacgaaacacc
AAAAAAAAAAAAAAAAAAAAAAAAAAAAAAAAAAAAAAAAAAAAAAAAAAAAAA
AAAAAAAAAAAAAAAAAAAAAGCTAAAAATGTATATGCCCAAAGcaagttgtggaaggtcca
gttttatgtcttcctgggacgaagacaagttgtggaaggtccagttttgaggggctattacaacttttttggtaccgagctcgaattcactggccg
tcgttttacaacgtcgtgactgggaaaaccctggcgttacccaacttaatcgccttgcagcacatccccctttcgccagctggcgtaatagcg
aagaggcccgcaccgatcgcccttcccaacagttgcgcagcctgaatggcgaatggcgcctgatgcggtattttctccttacgcatctgtgc
ggtatttcacaccgcatatggtgcactctcagtacaatctgctctgatgccgcatagttaagccagccccgacacccgccaacacccgctga
cgcgccctgacgggcttgtctgctcccggcatccgcttacagacaagctgtgaccgtctccgggagctgcatgtgtcagaggttttcaccgt
catcaccgaaacgcgcgagacgaaagggcctcgtgatacgcctatttttataggttaatgtcatgataataatggtttcttagacgtcaggtgg
cacttttcggggaaatgtgcgcggaacccctatttgtttatttttctaaatacattcaaatatgtatccgctcatgagacaataaccctgataaatg
cttcaataatattgaaaaaggaagagtatgagtattcaacatttccgtgtcgcccttattcccttttttgcggcattttgccttcctgtttttgctcacc
cagaaacgctggtgaaagtaaaagatgctgaagatcagttgggtgcacgagtgggttacatcgaactggatctcaacagcggtaagatcct
tgagagttttcgccccgaagaacgttttccaatgatgagcacttttaaagttctgctatgtggcgcggtattatcccgtattgacgccgggcaag
agcaactcggtcgccgcatacactattctcagaatgacttggttgagtactcaccagtcacagaaaagcatcttacggatggcatgacagtaa
gagaattatgcagtgctgccataaccatgagtgataacactgcggccaacttacttctgacaacgatcggaggaccgaaggagctaaccgc
ttttttgcacaacatgggggatcatgtaactcgccttgatcgttgggaaccggagctgaatgaagccataccaaacgacgagcgtgacacca
cgatgcctgtagcaatggcaacaacgttgcgcaaactattaactggcgaactacttactctagcttcccggcaacaattaatagactggatgg
aggcggataaagttgcaggaccacttctgcgctcggcccttccggctggctggtttattgctgataaatctggagccggtgagcgtgggtct
cgcggtatcattgcagcactggggccagatggtaagccctcccgtatcgtagttatctacacgacggggagtcaggcaactatggatgaac
gaaatagacagatcgctgagataggtgcctcactgattaagcattggtaactgtcagaccaagtttactcatatatactttagattgatttaaaac
ttcatttttaatttaaaaggatctaggtgaagatcctttttgataatctcatgaccaaaatcccttaacgtgagttttcgttccactgagcgtcagac
cccgtagaaaagatcaaaggatcttcttgagatcctttttttctgcgcgtaatctgctgcttgcaaacaaaaaaaccaccgctaccagcggtgg
ttttttgccggatcaagagctaccaactctttttccgaaggtaactggcttcagcagagcgcagataccaaatactgttcttctagtgtagccgt
agttaggccaccacttcaagaactctgtagcaccgcctacatacctcgctctgctaatcctgttacc
poly(A) tail (30 nt)
SEQ ID NO: 5
AAAAAAAAAAAAAAAAAAAAAAAAAAAAAA
  poly(A) tail (50 nt)
SEQ ID NO: 6
AAAAAAAAAAAAAAAAAAAAAAAAAAAAAAAAAAAAAAAAAAAAAAAAAA
poly(A) tail (75 nt)
SEQ ID NO: 7
AAAAAAAAAAAAAAAAAAAAAAAAAAAAAAAAAAAAAAAAAAAAAAAAAAAAAAAAAAAAAAAAAAAAAAAAAAA
gRNA (MeCP2-g6)
SEQ ID NO: 8
GCTAAAAATGTATATGCCCAAAG
```

### Poly(A) Tail

As used herein, a “poly(A) tail” refers to stretch of adenosine residues located at the end of an mRNA. In wild-type situations, a poly(A) tail is located at the 3′ end of a mRNA, and is post-transcriptionally synthesized on mRNAs that include a polyadenylation (poly(A)) signal sequence. The term “poly(A) signal sequence” or “poly(A) signal” is a sequence that triggers the endonuclease cleavage of a mRNA and the addition of a sequence of adenosine to the 3′end of the cleaved mRNA. Non-limiting examples of poly(A) signals include: bovine growth hormone (bGH) poly(A) signal, human growth hormone (hGH) poly(A) signal. Additional examples of poly(A) signal sequences are known in the art.

Poly(A) tails function by binding poly(A) binding protein (PABP). PABP is a highly conserved RNA binding protein in eukaryotes. This protein has four N-terminal RNA recognition motif (RRM) domains, which bind poly(A) RNA with a nanomolar affinity. The RRMs are followed by a proline-rich linker and a C-terminal MLLE domain. The MLLE domain recognizes a peptide motif called poly(A)-interacting motif 2 (PAM2), which is found in a number of PABP partner proteins that regulate mRNA metabolism (stability and translation). The presence of PABP on mRNA is known to stimulate their activity, enhancing translation and mRNA stability.

Poly(A) tails are added to most nascent eukaryotic messenger RNAs (mRNAs) at their 3′ end during a complex process that includes cleavage of the primary transcript and a coupled polyadenylation reaction driven by the poly(A) sisignal sequence. The term “polyadenylation” refers to the covalent linkage of a polyadenylyl moiety, or its modified variant, to the 3′ end of an mRNA molecule. A poly(A) tail is a long sequence of adenine nucleotides (e.g., 40, 50, 100, 200, 500, 1000) added to the pre-mRNA by a polyadenylate polymerase. In some embodiments, a gene delivery vector can include a sequence encoding a poly(A) signal sequence, such that the poly(A) signal sequence directs polyadenylation of an encoded guide RNA.

In some embodiments, a gene delivery vector can include a sequence encoding a poly(A) tail proximal to a sequence encoding a guide RNA. In some embodiments, a gene delivery vector can include a poly(T) sequence proximal to a sequence encoding a guide RNA, wherein the poly(T) sequence encodes a poly(A) tail. In some embodiments, a gene delivery vector can include a sequence comprising a poly(A) tail at the end of an isolated nucleic acid encoding a guide RNA.

In some embodiments, the poly(A) tail is located at the 3′ end of the gRNA. In some embodiments, the poly(A) tail is located at the 5′ end of the gRNA.

In some embodiments, the poly(A) tail comprises about 20, 25, 30, 35, 40, 45, 50, 55, 60, 65, 70, 75, 80, 85, 90, 95, 100, 200, 300, 400, 500, 600, 700, 800, 900, 1000, or more nucleotides, or any number of nucleotides between these values. In some embodiments, the poly(A) tail comprises about 30 nucleotides. In some embodiments, the poly(A) tail comprises about 50 nucleotides. In some embodiments, the poly(A) tail comprises about 75 nucleotides.

### Therapeutic Applications

In some embodiments, provided herein are methods of treating or preventing a disorder in a subject by administering any one of the recombinant expression systems or any one of the expression vectors described herein to the subject. In some embodiments, the disorder is a haploinsufficiency disorder.

Haploinsufficiency occurs when one gene allele is inactivated and the amount of gene product expressed from the remaining active allele is insufficient for proper gene function. A number of disorders are associated with, or are caused by haploinsufficiency.

In some embodiments, the haploinsufficiency disorder is selected from 5qsyndrome, Adams-Oliver syndrome 1, Adams-Oliver syndrome 3, Adams-Oliver syndrome 5, Adams-Oliver syndrome 6, Alagille syndrome 1, Autoimmune lymphoproliferative syndrome type IA, Autoimmune lymphoproliferative syndrome type V, Autosomal dominant deafness-2A, Brain malformations with or without urinary tract defects (BRMUTD), Carney complex type 1, CHARGE syndrome, Cleidocranial dysplasia, Currarino syndrome, Denys-Drash syndrome/Frasier syndrome, Developmental delay, intellectual disability, obesity, and dysmorphic features (DIDOD), DiGeorge syndrome (TBXI-associated), Dravet syndrome, Duane-radial raysyndrome, Ehlers-Danlos syndrome (classic-like), Ehlers-Danlos syndrome (vascular type), Feingold syndrome 1, Frontotemporal lobar degeneration with TDP43 inclusions (FTLD-TDP), GRN-related, GLUT I deficiency syndrome, Greig cephalopolysyndactyly syndrome, Hereditary hemorrhagic telangiectasia type 1, Holoprosencephaly 3, Holoprosencephaly 4, Holoprosencephaly 5, Holt-Oram syndrome, Hypoparathyroidism, sensorineural deafness, andrenal disease (HDR), Kleefstra syndrome 1, Klippel-Trenaunay syndrome (AAGF-related), Leri-Weill dyschondrosteosis, Marfan syndrome, Mental retardation and distinctive facial features with or without cardiac defects (MRFACD), Mental retardation, autosomal dominant 1, Mental retardation, autosomal dominant 19, Mental retardation, autosomal dominant 29, Nail-patella syndrome (NPS), Phelan-McDermid syndrome, Pitt-Hopkins syndrome, Primary pulmonary hypertension 1, Rett syndrome (congenital variant), Smith-Magenis syndrome (RAII associated), Sotos syndrome 1, Sotos syndrome 2, Stickler syndrome type I, Supravalvular aorticstenosis, SYNGAPI-related intellectual disability, Treacher Collins syndrome, Trichorhinophalangeal syndrome type I, Ulnar-mammary syndrome, van der Woude syndrome1, Waardenburg syndrome type 1, W aardenburg syndrome type 2A, and Waardenburg syndrometype 4C.

In some embodiments, the haploinsufficient gene is selected from the group consisting of AGGF1, ARHGAP31, BMPR2, CHD7, COL2A1, COL3A1, CTLA4, CTNNBI, DLL4, EHMTI, ELN, ENG, FAS, FBNI, FOXGI, GATA3, GLI3, GRN, IRF6, JAGI, KCNQ4, LMXIB, MBD5, MED13L, MITF, MNXI, MYCN, NFIA, NFIX, NOTCH!, NSDI, PAX3, PHIP, PRKARIA, RAil, RBPJ, RPS14, RUNX2, SALL4, SCNIA, SETBPI, SHANK3, SHH, SHOX, SLC2AI/GLUT1, SOXIO, SYNGAPI, TBXI, TBX3, TBX5, TCF4, TCOFI, TGIFI, TNXB, TRPSI, WTI, ZIC2, and combinations thereof.

In some embodiments, the haploinsufficiency disorder and haploinsufficient gene combination is a combination shown in Table 1.

TABLE 1
Haploinsufficiency disorders and genes.

| | Haploinsufficient |
|---|---|
| Haploinsufficiency Disorder | Gene |
| 5q-syndrome | RPS14 |
| Adams-Oliver syndrome I | ARHGAP31 |
| Adams-Oliver syndrome 3 | RBPJ |
| Adams-Oliver syndrome 5 | NOTCH1 |
| Adams-Oliver syndrome 6 | DLL4 |
| Alagille syndrome I | JAG1 |
| Autoimmune lymphoproliferative syndrome type IA | FAS |
| Autoimmune lymphoproliferative syndrome type V | CTLA4 |
| Autosomal dominant deafness-2A | KCNQ4 |
| Brain malformations with or without urinary tract defects (BRMUTD) | NFIA |
| Carney complex type 1 | PRKARIA |
| CHARGE syndrome | CHD7 |
| Cleidocranial dysplasia | RUNX2 |
| Currarino syndrome | MNX1 |
| Denys-Drash syndrome/ Frasier syndrome | WT1 |
| Developmental delay, intellectual disability, obesity, and dysmorphic | PHIP |
| features (DIDOD) | |
| DiGeorge syndrome (TBXI-associated) | TBX1 |
| Dravet syndrome | SCNIA |
| Duane-radial ray syndrome | SALL4 |
| Ehlers-Danlos syndrome (classic-like) | TNXB |
| Ehlers Danlos syndrome (vascular type) | COL3 A1 |
| Feingold syndrome 1 | MYCN |
| Frontotemporal lobar degeneration with TDP43 inclusions (FTLD-TDP), | GRN |
| GRN-related | |
| GLUT1 deficiency syndrome | SLC2A1 |
| Greig cephalopolysyndactyly syndrome | GLI3 |
| Hereditary hemorrhagic telangiectasia type 1 | ENG |
| Holoprosencephaly 3 | SHH |
| Holoprosencephaly 4 | TGIF1 |
| Holoprosencephaly 5 | ZIC2 |
| Holt-Oram syndrome | TBX5 |
| Hypoparathyroidism, sensorineural deafness, and renal disease (HDR) | GATA3 |
| Kleefstra syndrome 1 | EHMTI |
| Klippel-Trenaunay syndrome (AAGF-related) | AGGF1 |
| Leri-Weill dyschondrosteosis | SHOX |
| Marfan syndrome | FBN1 |
| Mental retardation and distinctive facial features with or without | MED13L |
| cardiac defects (MRFACD) | |
| Mental retardation, autosomal dominant 1 | MBD5 |
| Mental retardation, autosomal dominant 19 | CTNNB1 |
| Mental retardation, autosomal dominant 29 | SETBP1 |
| Nail-patella syndrome (NPS) | LMXIB |
| Phelan-McDermid syndrome | SHANK3 |
| Pitt-Hopkins syndrome | TCF4 |
| Primary pulmonary hypertension 1 | BMPR2 |
| Rett syndrome (congenital variant) | FOXGI |
| Smith-Magenis syndrome (RAIl-associated) | RAI1 |
| Sotos syndrome 1 | NSD1 |
| Sotos syndrome 2 | NFIX |
| Stickler syndrome type I | COL2A1 |
| Supravalvular aortic stenosis | ELN |
| SYNGAPI-related intellectual disability | SYNGAPI |
| Treacher Collins syndrome | TCOF1 |
| Trichorhinophalangeal syndrome type I | TRPSI |
| Ulnar-mammary syndrome | TBX3 |
| van der Woude syndrome 1 | IRF6 |
| Waardenburg syndrome type 1 | PAX3 |
| Waardenburg syndrome type 2A | MITF |
| Waardenburg syndrome type 4C | SOXIO |

### CNS Haploinsufficiency Disorders

In some embodiments, the haploinsufficiency disorder is a CNS haploinsufficiency disorder. In some embodiments, the haploinsufficiency disorder is selected from the group consisting of episodic ataxia, familial hemiplegia migraine, CDKL5 deficiency disorder, CHD2 myoclonic encephalopathy, familial focal epilepsy with variable loci, FOXGI syndrome, benign familial neonatal seizures, Rett syndrome, Dravat syndrome, SCN2A-epileptic encephalopathy, SCN2A-developmental encephalopathy, SCN8A-epileptic encephalopathy, SC8A familial infantile epilepsy, early infantile epileptic encephalopathy, myoclonic-atonic epilepsy, early infantile epileptic encephalopathy, SYNGAP1-related intellectual disability, tuberous sclerosis, Lennox-Gastaut Syndrome, FoxG1 syndrome, KCNQ2-related epileptic encephalopathy, PCDH19-related epilepsy, SLC6A1-related myoclonic-astatic epilepsy, STXBP1-related epileptic encephalopathy, SYNGAP1 syndrome, and combinations thereof.

In some embodiments, the haploinsufficiency gene is selected from the group consisting of SCN1A, SCN2A, SCN8A, SCN12A5, SPTAN1, CDKL5, CHD2, FOXG1, KCNQ2, PCDH19, SLC6A1, STXBP1, SYNGAP1, CACNA1A, DEPDC5, MECP2, TSC1, TSC2, and combinations thereof.

In some embodiments, the haploinsufficiency disorder and haploinsufficient gene combination is a combination shown in Table 2

TABLE 2
CNS haploinsufficiency disorders and genes

| Haploinsufficiency Disorder | Haploinsufficient Gene |
|---|---|
| Episodic ataxia, Familial hemiplegia migraine | CACNA1A |
| CDKL5 deficiency disorder | CDKL5 |
| CHD2 myoclonic encephalopathy | CHD2 |
| Lennox-Gastaut Syndrome | CHD2 |
| Familial focal epilepsy with variable loci | DEPDC5 |
| FOXG1 syndrome | FOXG1 |
| Benign familial neonatal seizures | KCNQ2 |
| Rett syndrome | MECP2 |
| Dravet syndrome | SCNIA |
| SCN2A-epileptic encephalopathy | SCN2A |
| SCN2A-developmental encephalopathy | SCN2A |
| SCN8A-epileptic encephalopathy | SCN8A |
| SCN8A familial infantile epilepsy | SCN8A |
| Early infantile epileptic encephalopathy | SLC12A5 |
| Myoclonic-atonic epilepsy | SLC6A1 |
| Early infantile epileptic encephalopathy | SPTAN1 |
| SYNGAP1-related intellectual disability | SYNGAP1 |
| Tuberous sclerosis | TSC1 |
| Tuberous sclerosis | TSC2 |
| KCNQ2-related epileptic encephalopathy | KCNQ2 |
| SLC6A1-related myoclonic-astatic epilepsy | SLC6A1 |
| STXBP1-related epileptic encephalopathy | STXBP1 |
| SYNGAP1 syndrome | SYNGAP1 |

### EXAMPLES

The disclosure is further described in the following examples, which do not limit the scope of the disclosure described in the claims.

### Example 1—Increase in mRNA Translation with dCas13b and mRNA Specific gRNA

The human neuroblastoma cell line SH-SY5Y were cultured in Dulbecco's modified Eagle's medium (Life Technologies, USA). The medium was supplemented with 10% fetal bovine serum, 1× nonessential amino acids, and 2 mM L-glutamine. Cells were incubated at 37° C. in 5% CO₂ and their medium has changed every 72 hours. To transfect SH-Sy5Y cells, GeneXPlus (ATCC® ACS-4004) transfection reagent were utilized following the company instruction. Cells incubated at 37° C. in 5% CO₂ for 48 hours post transfection. The whole cell lysate and total RNA extracted using RIPA buffer and Trizol/chloroform respectively.

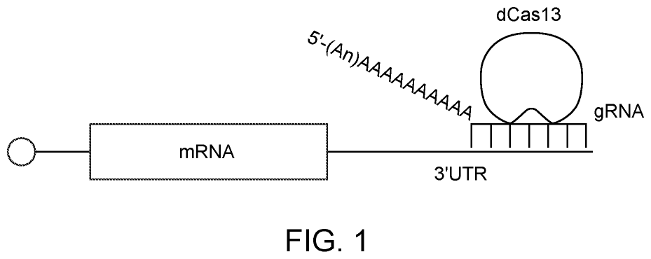

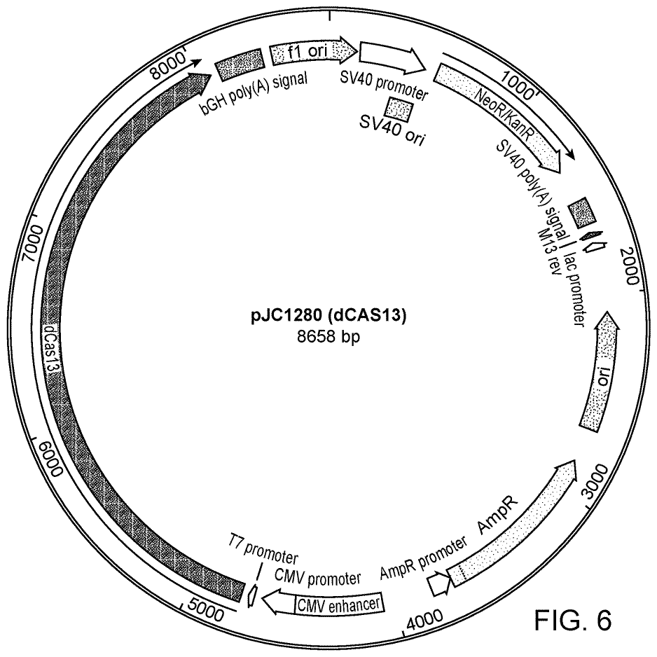

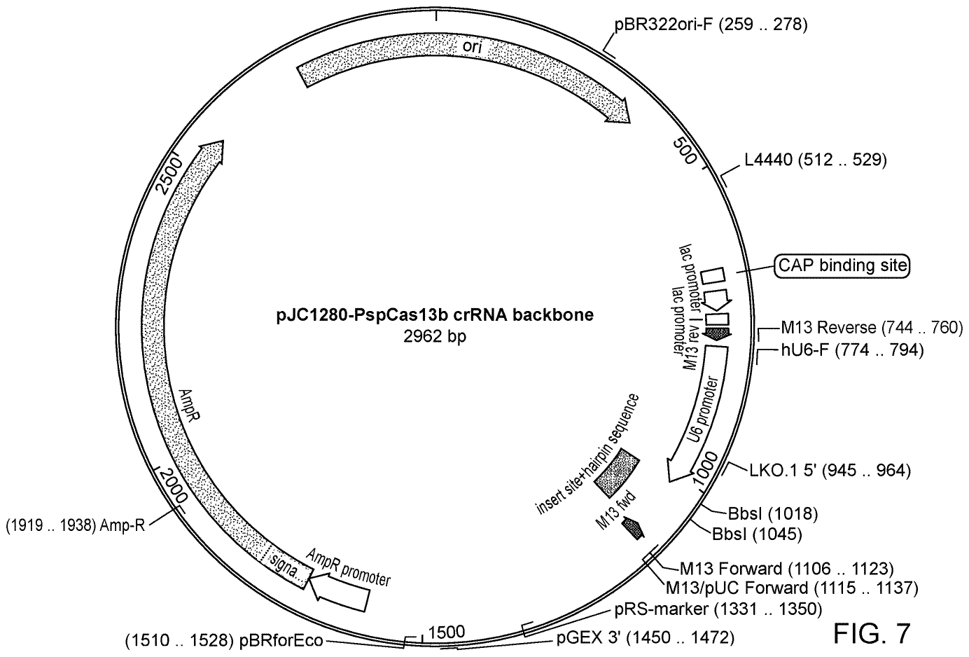

To prepare poly(A) tail+guide RNA construct (FIG. 1), the optimal guide RNA against 3′UTR of MeCP2 (GCTAAAAATGTATATGCCCAAAG (SEQ ID NO: 8)) namely Mecp2-g6 was screened and selected. Oligos with different polyA tail consist of 30, 50 and 75 A nt in the 5′ end of guide RNA was designed and synthesized by IDT (Integrated DNA Technologies). The synthesized oligos were cloned in pJc1208 (PspCas13b crRNA backbone) using the Gibson assembly strategy (FIG. 6). The SH-SY5Y cells were co-transfected with either of pJC1276 (30A Mecp2-g6), pJC 1277 (50A Mecp2-g6) or pJC 1278 (75A Mecp2-g6) along with dCas 13b expressing vector (pJC 1280) (FIG. 7). The empty vector (pJC1208) plus pJC1280 was employed as a control.

5 Western blot analysis was performed on the whole cell extract and the MeCP2 protein level was shown using 1:1000 dilution of MeCP2 (D4F3) XP® Rabbit mAb 3456 cell signaling antibody. Obtained results were quantified and normalized with the level of GAPDH protein.

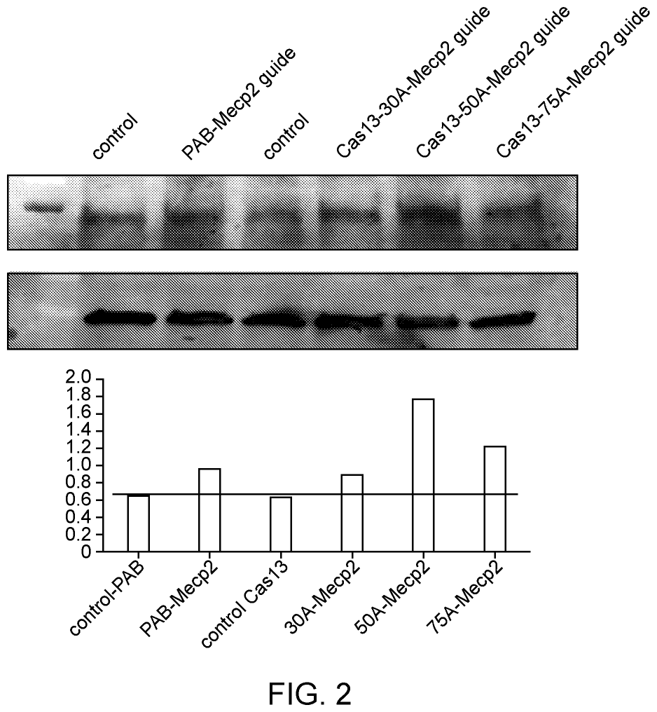

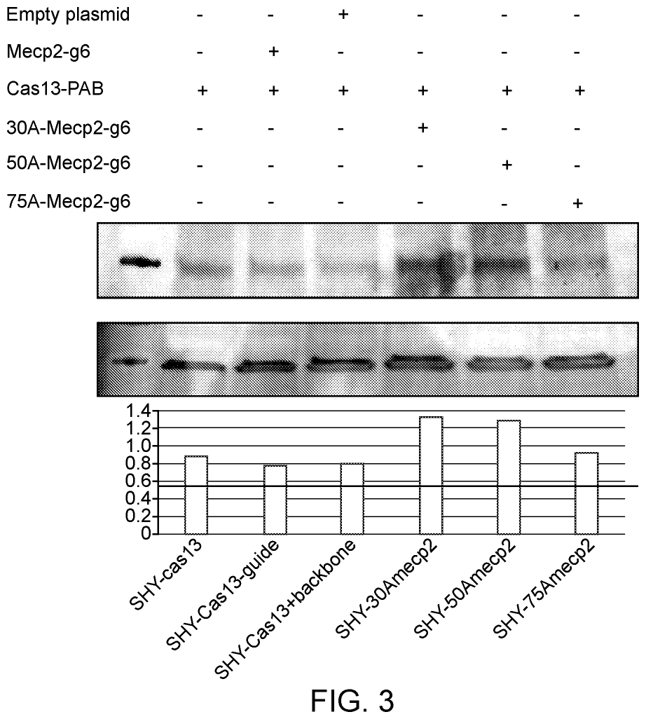

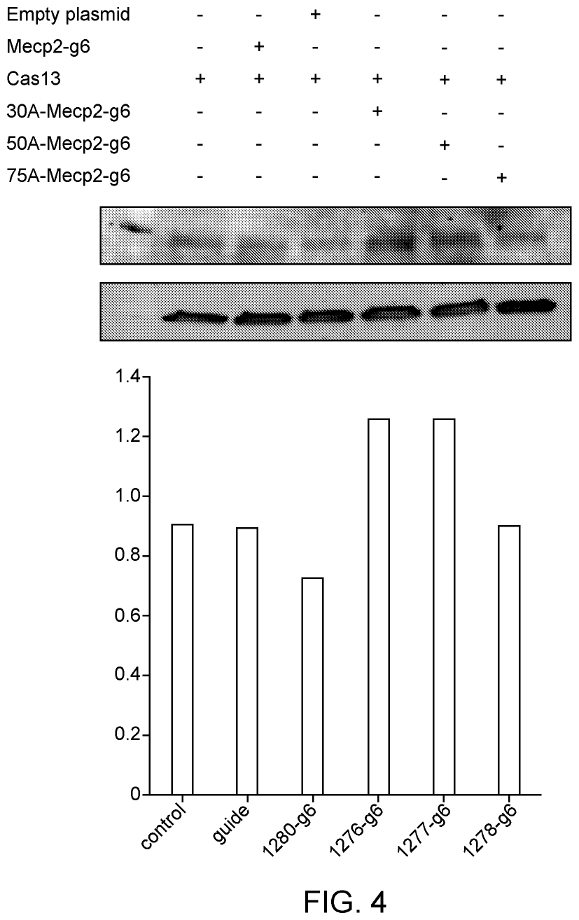

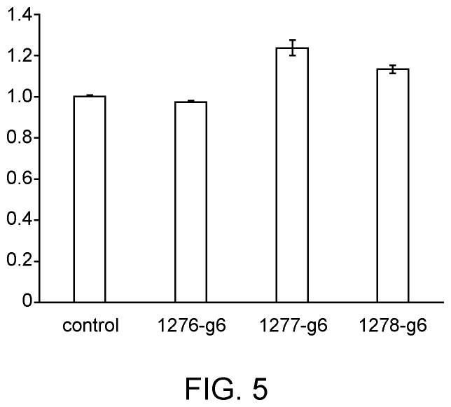

The cDNA synthesis proceeded for the RNA extract and qRT-PCR performed using the PowerUp SYBER Green Master Mix (applied biosystem) and primer set for MeCP2 mRNA was designed. The results of three biological repeats demonstrated increase in the level of Mecp2 after transfection with either of 30 A or 50 A-mecp2 guide RNA (FIGS. 2-5).

What is claimed is:

1. A system comprising:
(a) an RNA binding moiety; and
(b) a gRNA hybrid comprising (i) a complementary region that hybridizes with an mRNA of an active allele of a gene associated with a disorder associated with a decrease in the expression of a protein from the mRNA and (ii) a poly(A) tail;
wherein the gRNA hybrid forms a complex with the RNA binding moiety.

2. The system of claim 1, wherein the RNA binding moiety is a Cas protein selected from the group consisting of Cas9, Cas12, Cas13, and Cas14.

3. The system of claim 1 or claim 2, wherein the RNA binding moiety is Cas13b.

4. The system of any one of claims 1-3, wherein the RNA binding moiety is a catalytically inactive Cas protein.

5. The system of any one of claims 1-4, wherein the poly(A) tail is located at the 5′ end of the gRNA hybrid.

6. The system of any one of claims 1-4, wherein the poly(A) tail is located at the 3′ end of the gRNA hybrid.

7. The system of any one of claims 1-6, wherein the poly(A) tail comprises about 30 nucleotides.

8. The system of any one of claims 1-6, wherein the poly(A) tail comprises about 50 nucleotides.

9. The system of any one of claims 1-6, wherein the poly(A) tail comprises about 75 nucleotides.

10. The system of any one of claims 1-9, wherein the disorder is a haploinsufficiency disorder.

11. The system of any one of claims 1-10, wherein the RNA binding moiety is encoded by a sequence that comprises or consists of SEQ ID NO: 1 or a sequence that has at least 80%, at least 85%, at least 90%, at least 95%, or at least 99% sequence identity to SEQ ID NO: 1.

12. The system of any one of claims 1-11, wherein the gRNA hybrid is encoded by a sequence comprising or consisting of SEQ ID NO: 2, SEQ ID NO: 3, or SEQ ID NO: 4.

13. A recombinant expression system comprising:
(a) a nucleic acid sequence encoding an RNA binding moiety; and
(b) a nucleic acid sequence encoding a gRNA hybrid comprising (i) a complementary region that hybridizes with an mRNA of an active allele of a gene associated with a disorder associated with a decrease in the expression of a protein from the mRNA and (ii) a poly(A) tail;
wherein the gRNA hybrid forms a complex with the RNA binding moiety.

14. The recombinant expression system of claim 13, wherein the RNA binding moiety is a Cas protein selected from the group consisting of Cas9, Cas12, Cas13, and Cas14.

15. The recombinant expression system of claim 13 or claim 14, wherein the RNA binding moiety is Cas13b.

16. The recombinant expression system of any one of claims 13-15, wherein the RNA binding moiety is a catalytically inactive Cas protein.

17. The recombinant expression system of any one of claims 13-16, wherein the poly(A) tail is located at the 5′ end of the gRNA hybrid.

18. The recombinant expression system of any one of claims 13-16, wherein the poly(A) tail is located at the 3′ end of the gRNA hybrid.

19. The recombinant expression system of any one of claims 13-18, wherein the poly(A) tail comprises about 30 nucleotides.

20. The recombinant expression system of any one of claims 13-18, wherein the poly(A) tail comprises about 50 nucleotides.

21. The recombinant expression system of any one of claims 13-18, wherein the poly(A) tail comprises about 75 nucleotides.

22. The recombinant expression system of any one of claims 13-21, wherein the disorder is a haploinsufficiency disorder.

23. The recombinant expression system of any one of claims 13-22, wherein the RNA binding moiety is encoded by a sequence that comprises or consists of SEQ ID NO: 1 or a sequence that has at least 80%, at least 85%, at least 90%, at least 95%, or at least 99% sequence identity to SEQ ID NO: 1.

24. The recombinant expression system of any one of claims 13-23, wherein the gRNA hybrid is encoded by a sequence comprising or consisting of SEQ ID NO: 2, SEQ ID NO: 3, or SEQ ID NO: 4.

25. An expression vector comprising the recombinant expression system of any one of claims 13-24.

26. The expression vector of claim 25, wherein the expression vector is a viral vector.

27. The expression vector of claim 26, wherein the viral vector is an adeno-associated viral vector (AAV), a lentiviral vector, or an adenoviral vector.

28. A cell comprising the recombinant expression system of any one of claims 13-24 or the expression vector of any one of claims 25-27.

29. A method of treating or preventing a haploinsufficiency disorder in a subject, the method comprising:
administering the recombinant expression system of any one of claims 13-24 or the expression vector of any one of claims 25-27 to the subject.

30. The method of claim 29, wherein the haploinsufficiency disorder is selected from the group consisting from 5qsyndrome, Adams-Oliver syndrome 1, Adams-Oliver syndrome 3, Adams-Oliver syndrome 5, Adams-Oliver syndrome 6, Alagille syndrome 1, Autoimmune lymphoproliferative syndrome type IA, Autoimmune lymphoproliferative syndrome type V, Autosomal dominant deafness-2A, Brain malformations with or without urinary tract defects (BRMUTD), Carney complex type 1, CHARGE syndrome, Cleidocranial dysplasia, Currarino syndrome, Denys-Drash syndrome/Frasier syndrome, Developmental delay, intellectual disability, obesity, and dysmorphic features (DIDOD), DiGeorge syndrome (TBXI-associated), Dravet syndrome, Duane-radial raysyndrome, Ehlers-Danlos syndrome (classic-like), Ehlers-Danlos syndrome (vascular type), Feingold syndrome 1, Frontotemporal lobar degeneration with TDP43 inclusions (FTLD-TDP), GRN-related, GLUT I deficiency syndrome, Greig cephalopolysyndactyly syndrome, Hereditary hemorrhagic telangiectasia type 1, Holoprosencephaly 3, Holoprosencephaly 4, Holoprosencephaly 5, Holt-Oram syndrome, Hypoparathyroidism, sensorineural deafness, andrenal disease (HDR), Kleefstra syndrome 1, Klippel-Trenaunay syndrome (AAGF-related), Leri-Weill dyschondrosteosis, Marfan syndrome, Mental retardation and distinctive facial features with or without cardiac defects (MRFACD), Mental retardation, autosomal dominant 1, Mental retardation, autosomal dominant 19, Mental retardation, autosomal dominant 29, Nail-patella syndrome (NPS), Phelan-McDermid syndrome, Pitt-Hopkins syndrome, Primary pulmonary hypertension 1, Rett syndrome (congenital variant), Smith-Magenis syndrome (RAII associated), Sotos syndrome 1, Sotos syndrome 2, Stickler syndrome type I, Supravalvular aorticstenosis, SYNGAPI-related intellectual disability, Treacher Collins syndrome, Trichorhinophalangeal syndrome type I, Ulnar-mammary syndrome, van der Woude syndrome1, Waardenburg syndrome type 1, W aardenburg syndrome type 2A, and Waardenburg syndrometype 4C.

31. The method of claim 29, wherein the haploinsufficiency disorder is a CNS haploinsufficiency disorder.

32. The method of claim 31, wherein the CNS haploinsufficiency disorder is selected from the group consisting of episodic ataxia, familial hemiplegia migraine, CDKL5 deficiency disorder, CHD2 myoclonic encephalopathy, familial focal epilepsy with variable loci, FOXG1 syndrome, benign familial neonatal seizures, Rett syndrome, Dravat syndrome, SCN2A-epileptic encephalopathy, SCN2A-developmental encephalopathy, SCN8A-epileptic encephalopathy, SC8A familial infantile epilepsy, early infantile epileptic encephalopathy, myoclonic-atonic epilepsy, early infantile epileptic encephalopathy, SYNGAP1-related intellectual disability, tuberous sclerosis, Lennox-Gastaut Syndrome, FoxG1 syndrome, KCNQ2-related epileptic encephalopathy, PCDH19-related epilepsy, SLC6A1-related myoclonic-astatic epilepsy, STXBP1-related epileptic encephalopathy, SYNGAP1 syndrome, and combinations thereof.

33. The method of any one of claims 29-32, wherein the subject is a mammal.

34. The method of claim 33, wherein the subject is a human.
